(19) United States
(12) Patent Application Publication
(10) Pub. No.: US 2026/0263617 A1
(43) Pub. Date: Sep. 10, 2026

(54) DARATUMUMAB DRUG CONJUGATES AND PREPARATION THEREOF

(71) Applicant: ZYDUS LIFESCIENCES LIMITED, Ahmedabad, Gandhinagar (IN)

(72) Inventors: Sanjeev Kumar MENDIRATTA, Ahmedabad, Gandhinagar (IN); Sanjay BANDYOPADHYAY, Ahmedabad, Gandhinagar (IN); Avanish SINGH, Ahmedabad, Gandhinagar (IN); Pankaj KALITA, Ahmedabad, Gandhinagar (IN); Swagat SONI, Ahmedabad, Gandhinagar (IN)

(73) Assignee: ZYDUS LIFESCIENCES LIMITED, Ahmedabad, Gandhinagar, GJ (IN)

(21) Appl. No.: 19/166,341

(22) PCT Filed: Mar. 16, 2024
(86) PCT No.: PCT/IB2024/052567

§ 371 Date: Sep. 17, 2025

(30) Foreign Application Priority Data
Mar. 17, 2023 (IN) 202321018199

(51) Int. Cl. A61K 47/68; A61K 45/06; A61P 35/00

(52) U.S. Cl. CPC A61K 47/6849; A61K 45/06; A61K 47/68031; A61K 47/6889; A61P 35/00

(57) ABSTRACT

The invention provides antibody-linker-drug conjugates wherein antibody is daratumumab and drug is auristatin analogues or a pharmaceutically acceptable salt thereof. The invention also provides a process of preparing antibody-linker-conjugates of the present invention.

### FIELD OF THE INVENTION

The invention provides antibody-linker-drug conjugates wherein antibody is daratumumab and drug is auristatin analogues.

### BACKGROUND OF THE INVENTION

Antibody drug conjugates (ADC's), composed of an antibody, linker, and cytotoxic agent (drug), are one of the most complex drug platforms in the oncology armamentarium ^{[1]}. They are a payload delivery system with major variables influencing their success including: (i) the rate of internalization of the payload; (ii) the expression of the target antigen on the tumor and normal tissues with the implications for both patient selection as well as therapeutic index; (iii) the linker chemistry and, implicit within the choice of chemistry, the extracellular as well as intracellular stability; and (iv) the selection of payload for the tumor indication ^{[2]}. The mechanisms of action of ADCs include direct binding between the antibody and its targeted cell surface antigen, and intracellular or extracellular release of cytotoxic drugs ^{[3]}. In 2015, Daratumumab, a CD38 antibody, was approved by the U.S. Food and Drug Administration (FDA) for the treatment of multiple myeloma ^{[4,5]}. CD38 is a 46-kDa type II transmembrane glycoprotein with a short 20-aa N-terminal cytoplasmic tail and a long 256-aa extracellular domain ^{[6]}. Functions ascribed to CD38 include receptor-mediated adhesion and signaling events, as well as important bifunctional ectoenzymatic activities that contribute to intracellular calcium mobilization ^{[7]}. Daratumumab binds to CD38, which is overexpressed on the surface of multiple myeloma cells, and consequently induces the tumor cell death by complement-dependent cytotoxicity (CDC), antibody-dependent cell-mediated cytotoxicity (ADCC), antibody-dependent cellular phagocytosis (ADCP), and other mechanisms directly associated with the functional change of surface antigen after binding. Present invention provides daratumumab derived antibody drug conjugates to enable specific delivery of drug (auristatin and/or its analogues) to CD38 overexpressed cancer cells.

### SUMMARY OF THE INVENTION

The invention provides antibody-linker-drug conjugates wherein antibody is daratumumab and drug is auristatin analogues. The invention also provides a process of preparing antibody-linker-conjugates of the present invention. The present invention is also directed to antibody-linker-drug conjugates in the treatment of cancer, an autoimmune disease or an infectious disease.

### ABBREVIATIONS

Ab: antibody
ADC: Antibody Drug Conjugate
HP-SEC: High performance size-exclusion chromatography
IRS: Internal Reference standard
MMAE: Monomethyl auristatin E
MMAF: Monomethyl auristatin F
PAB: para amino benzyl
PABC: para amino benzyl carbamate
SDS PAGE: Sodium dodecyl-sulfate polyacrylamide gel electrophoresis
TCEP: Tris (2-carboxyethyl) phosphine

### EMBODIMENTS OF THE INVENTION

### Embodiment 1

In one embodiment, the present invention provides antibody-linker-drug conjugates of Formula I: Ab [symbol] Aa-Ww-Yy-D)ₚ wherein antibody (Ab) is daratumumab; -Aa-Ww-Yy- is an enzymatically cleavable linker unit that links the drug unit and the antibody, wherein: -A- is a stretcher unit; a is 1; each—W— is independently an amino acid unit; —Y— is a spacer unit; w is an integer ranging from 2 to 12, y is 1 or 2; p ranges from 1 to about 20; and drug (D) is selected from MMAE and MMAF as defined by following structures;

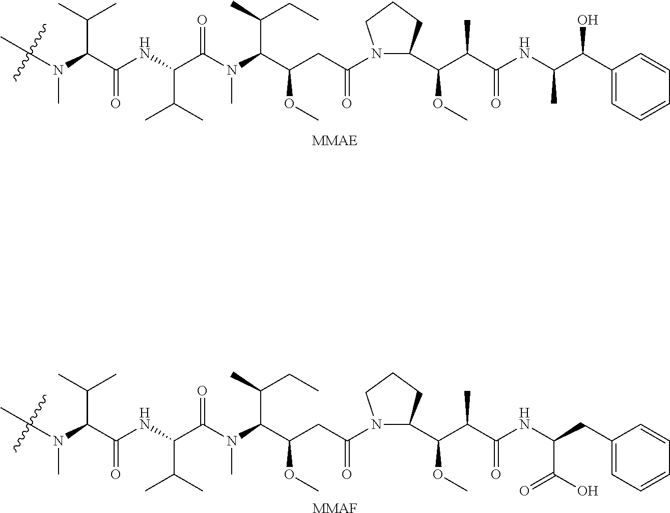

In further embodiment, the present invention provides antibody-linker-drug conjugates of formula Ab [symbol] Aa-Ww-Yy-D)ₚ wherein antibody (Ab) is daratumumab; -Aa-Ww-Yy- is an enzymatically cleavable linker unit that links the drug unit and the antibody, wherein: -A- is a stretcher unit; a is 1; each —W— is independently an amino acid unit selected from -phenylalanine-lysine- or -valine-citrulline-; —Y— is a spacer unit selected from -glycine-glycine- or p-amino benzyl alcohol (PAB) or p-aminobenzyl carbamate (PABC); w is an integer ranging from 2 to 12, y is 1 or 2; p ranges from 1 to about 20; and drug (D) is selected from MMAE and MMAF as defined by following structures:

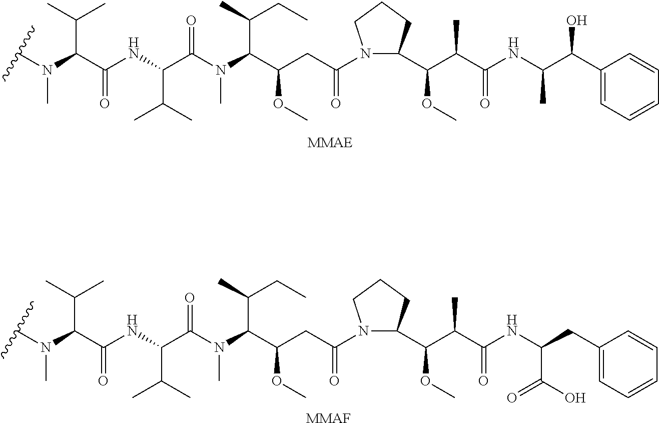

Wherein A is defined by following structure;

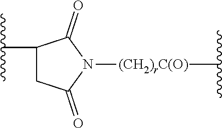

r is an integer ranging from 1 to 10; wherein carbonyl terminus of -A- forming bond with an amino acid unit (W) and the succinimido terminus of -A- forming bond with antibody (Ab).

In preferred embodiment, the present invention provides an antibody drug conjugate (ADC) structure represented by:

wherein mAb is a monoclonal antibody comprising light chain variable region complementarity determining region (CDR) sequences CDR1 (RASQSVSSYLA, SEQ ID NO: 1), CDR2 (DASNRAT, SEQ ID NO: 2) and CDR3 (QQRSNWPPTF, SEQ ID NO: 3) and heavy chain variable region CDR sequences CDR1 (SFAMS, SEQ ID NO: 4), CDR2 (AISGSGGGTYYADSVKG, SEQ ID NO: 5) and CDR3 (DKILWFGEPVFDY, SEQ ID NO: 6). In this embodiment, the antibody is daratumumab.

### Further Embodiments

In a second embodiment, the present invention provides a pharmaceutical composition comprising antibody drug conjugate, preferably daratumumab drug conjugate as embodied in embodiment 1 and an acceptable carrier. Acceptable carrier is defined as any suitable pharmaceutical excipient known in the art and which is compatible with active pharmaceutical ingredient (API), i.e. antibody drug conjugate, herein of the present invention.

In a third embodiment, the present invention provides a process for preparing the antibody drug conjugate, preferably daratumumab drug conjugate as embodied in embodiment 1 wherein process comprising a) purification of antibody daratumumab, b) partial reduction of daratumumab c) conjugation of the partially reduced daratumumab with vc-MMAE drug linker.

In a further embodiment, the daratumumab drug conjugate according to the present invention may be used in the treatment of cancer.

In a still further embodiment, the daratumumab drug conjugate according to the present invention may be used in the treatment of an autoimmune disease or an infectious disease.

In a further embodiment, the daratumumab drug conjugate according to the present invention may be used alone as a monotherapy in the treatment of cancer.

In a still further embodiment, the daratumumab drug conjugate according to the present invention may be used alone as a monotherapy in the treatment of an autoimmune disease or an infectious disease.

In a further embodiment, the daratumumab drug conjugate according to the present invention may be used in combination with other drug products in the treatment of cancer, an autoimmune disease or an infectious disease.

In further embodiment, the conjugate of the present invention may be administered either as an intravenous (i.v), intramuscular (i.m), subcutaneous (s.c.), intraperitoneal (i.p) or any other suitable route of administration.

In a further embodiment, the dose of the antibody drug conjugate (ADC) according to the present invention may be in the range of 0.1 mg/kg to 10 mg/kg.

In further embodiment, the present invention provides route of administration of daratumumab drug conjugate in a subject via intravenous (i.v), intramuscular (i.m), subcutaneous (s.c.), intraperitoneal (i.p) or any other suitable route of administration.

### DESCRIPTION OF FIGURES

FIG. 1: FIG. 1 demonstrate drug antibody ratio (DAR) of daratumumab-MMAE conjugates analysed by HP—HIC

FIG. 2: FIG. 2 demonstrates the polypeptide profile of daratumumab-MMAE conjugate of the present invention by SDS-PAGE analysis.

FIG. 3: FIG. 3 demonstrates in-vitro cytotoxicity assay of Daratumumab-MMAE conjugate using Daudi cells.

FIG. 4: FIG. 4 demonstrates efficacy of Daratumumab-ADC in Burkitt lymphoma xenograft disease model in SCID mice.

### DEFINITIONS

The term “linker” is any chemical moiety that is capable of linking a compound, usually a drug, such auristatin, to a cell-binding agent such daratumumab in covalent manner. The linker includes a stretcher unit (-A-), an amino acid unit (—W—), and a spacer unit (—Y—) as defined by formula;

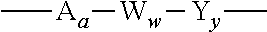

Linkers can be susceptible to or be substantially resistant to acid-induced cleavage, light-induced cleavage, peptidase-induced cleavage, esterase-induced cleavage, and disulfide bond cleavage, at conditions under which the compound or the antibody remains active. Suitable linkers are well known in the art and include, for example, disulfide groups, thioether groups, acid labile groups, photolabile groups, peptidase labile groups and esterase labile groups. Linkers also include charged linkers, and hydrophilic forms thereof as described herein and know in the art.

The term “pharmaceutical composition” refers to preparations which are in such form as to permit the biological activity of the active ingredients to be unequivocally effective, and which contain no additional components which are significantly toxic to the subjects to which the formulation would be administered. The term “pharmaceutical formulation”, “formulation”, “pharmaceutical composition”, or “composition” can be used here interchangeably.

The terms “patient” and “subject” are used interchangeably and are used in their conventional sense to refer to a living organism suffering from or prone to a condition that can be prevented or treated by administration of a composition of the present invention, and includes animals. The term “Animal” refers to a human or non-human animal, including, but not limited to, farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese and non-human primates, including, but not limited to, monkeys, chimpanzees and other apes and monkey species. The term does not denote a particular age. Thus, adult, juvenile and newborn individuals are of interest.

In the context of cancer, the term “treating” includes any or all of: preventing growth of tumor cells or cancer cells, preventing replication of tumor cells or cancer cells, lessening of overall tumor burden and ameliorating one or more symptoms associated with the disease.

In the context of an autoimmune disease, the term “treating” includes any or all of: preventing replication of cells associated with an autoimmune disease state including, but not limited to, cells capable of producing an autoimmune antibody, lessening the autoimmune-antibody burden and ameliorating one or more symptoms of an autoimmune disease.

In the context of an infectious disease, the term “treating” includes any or all of: preventing the growth, multiplication or replication of the pathogen that causes the infectious disease and/or pathogen-infected host cells and ameliorating one or more symptoms of an infectious disease.

The phrase “pharmaceutically acceptable salt,” as used herein, refers to pharmaceutically acceptable organic or inorganic salts of a drug (payload) of the present invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate salts.

Unless otherwise specified, a or an means “one or more.” An antibody drug conjugate (ADC) is defined as an antibody component or fragment thereof conjugated to a therapeutic agent. As used herein, the terms “antibody drug conjugate”, “antibody-linker-drug” “conjugate” and “immunoconjugate” are used interchangeably herein the present invention. The preferred antibody drug conjugate of the present invention is daratumumab-MMAE conjugate. The “daratumumab-MMAE conjugate” and “daratumumab vedotin” are used interchangeably.

### DETAILED DESCRIPTION OF THE PRESENT INVENTION

The present invention provides antibody-linker-drug conjugates of Ab [symbol] Aa-Ww-Yy-D)ₚ wherein antibody (Ab) is daratumumab; -Aa-Ww-Yy- is an enzymatically cleavable linker unit that links the drug unit and the antibody, wherein: -A- is a stretcher unit; a is 1; each —W— is independently an amino acid unit; —Y— is a spacer unit; w is an integer ranging from 2 to 12, y is 1 or 2; p ranges from 1 to about 20; and drug (D) is auristatin analogues or a pharmaceutically acceptable salt thereof.

### Antibody (Ab):

The antibody of the antibody drug conjugates (ADC) of the present invention is CD-38 targeting antibody, preferably daratumumab or any posttranslational modification of an antibody or antibody variant thereof. The posttranslational modification of the antibody includes, but not limited to, deamidated, acidic, basic or oxidized variants or any other variants of an antibody that do not impact the biological function or efficacy of the antibody. In one embodiment, the antibody of the antibody drug conjugates (ADC) of the present invention is daratumumab variants. Daratumumab variants of the present invention include an amino acid substitution, insertion, and/or deletion in a polypeptide sequence of the antibody daratumumab

In one embodiment, the antibody of the antibody drug conjugates (ADC) of the present invention is daratumumab variants. Daratumumab variants of the present invention includes an amino acid substitution, insertion, and/or deletion in a polypeptide sequence of the antibody daratumumab. In of the other embodiment, the CD-38 targeting antibody according to the present invention is an antibody that comprises light chain variable region complementarity determining region (CDR) sequences CDR1 (RASQSVSSYLA, SEQ ID NO: 1), CDR2 (DASNRAT, SEQ ID NO: 2) and CDR3 (QQRSNWPPTF, SEQ ID NO: 3) and heavy chain variable region CDR sequences CDR1 (SFAMS, SEQ ID NO: 4), CDR2 (AISGSGGGTYYADSVKG, SEQ ID NO: 5) and CDR3 (DKILWFGEPVFDY, SEQ ID NO: 6). The said sequence has been disclosed in International Nonproprietary Names for Pharmaceutical Substances (INN, Vol. 24, No. 1, 2010^{[8]} for daratumumab product. The antibody of the antibody drug conjugates (ADC) of the present invention is daratumumab and comprises light chain sequence (EIVLTQSPAT LSLSPGERAT LSCRASQSVS SYLAWYQQKP GQAPRLLIYD ASNRATGIPA RFSGSGSGTD FTLTISSLEP EDFAVYYCQQ RSNWPPTFGQ GTKVEIKRTV AAPSVFIFPP SDEQLKSGTA SVVCLLNNFY PREAKVQWKV DNALQSGNSQ ESVTEQDSKD STYSLSSTLT LSKADYEKHK VYACEVTHQG LSSPVTKSFN RGEC, SEQ ID NO: 7) and heavy chain (EVQLLESGGG LVQPGGSLRL SCAVSGFTFN SFAMSWVRQA PGKGLEWVSA ISGSGGGTYY ADSVKGRFTI SRDNSKNTLY LQMNSLRAED TAVYFCAKDK ILWFGEPVFD YWGQGTLVTV SSASTKGPSV FPLAPSSKST SGGTAALGCL VKDYFPEPVT VSWNSGALTS GVHTFPAVLQ SSGLYSLSSV VTVPSSSLGT QTYICNVNHK PSNTKVDKRV EPKSCDKTHT CPPCPAPELL GGPSVFLFPP KPKDTLMISR TPEVTCVVVD VSHEDPEVKF NWYVDGVEVH NAKTKPREEQ YNSTYRVVSV LTVLHQDWLN GKEYKCKVSN KALPAPIEKT ISKAKGQPRE PQVYTLPPSR EEMTKNQVSL TCLVKGFYPS DIAVEWESNG QPENNYKTTP PVLDSDGSFF LYSKLTVDKS RWQQGNVFSC SVMHEALHNH YTQKSLSLSP GK, SEQ ID NO: 8). The daratumumab is an immunoglobulin G1 kappa (IgG1κ) human monoclonal antibody against CD38 antigen, produced in a mammalian cell line (Chinese Hamster Ovary [CHO]) using recombinant DNA technology. The molecular weight of daratumumab is approximately 148 kDa. In one embodiment, the antibody of the antibody drug conjugates (ADC) of the present invention is daratumumab or its variants that can improve binding affinity of ADC to the CD38 antigen. In one more embodiment, the antibody of the antibody drug conjugates (ADC) of the present invention is daratumumab or its variants that can improve binding affinity of ADC to the CD38 antigen at suitable pH. The preferred pH can be pH 6.0 or pH 7.0. In another embodiment, the antibody of the present invention is daratumumab or its variants that can be used to increase the half-life of ADC.

### The Linker Unit:

The Linker unit of the Antibody-Linker-Drug Conjugate links the Drug unit and the antibody unit and has the formula:

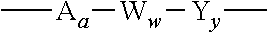

### Wherein:

-A- is a stretcher unit;
a is 0 or 1;
each —W— is independently an amino acid unit;
w is independently an integer ranging from 0 to 12;
—Y— is a spacer unit; and
Y is 0, 1 or 2.

The stretcher unit: The Stretcher unit (-A-), when present, links an antibody unit to an amino acid unit (—W—). In this regard antibody (Ab) has a functional group that can form a bond with a functional group of a Stretcher. Useful functional groups that can be present on an antibody, either naturally or via chemical manipulation include, but are not limited to, sulfhydryl (—SH), amino, hydroxyl, carboxy, the anomeric hydroxyl group of a carbohydrate, and carboxyl. Preferred antibody functional groups are sulfhydryl and amino.

The stretcher unit (-A-) of the present invention is defined by following structure;

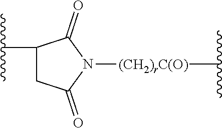

r is an integer ranging from 1 to 10; wherein carbonyl terminus of -A- forming bond with an amino acid unit (W) and the succinimido terminus of -A- forming bond with antibody (Ab).

In one embodiment, the Stretcher unit forms a bond with a sulfur atom of the antibody unit. The sulfur atom can be derived from a sulfhydryl group of antibody. Representative Stretcher units of this embodiment are depicted as below;

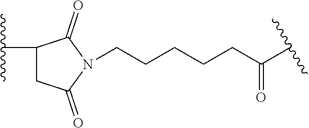 [SMILES: *C(=O)CCCCCN1C(=O)CC(*)C1=O]

The amino acid unit (—W—): The Amino Acid unit (—W—), when present, links the Stretcher unit to the Spacer unit if the Spacer unit is present, links the Stretcher unit to the Drug unit if the Spacer unit is absent, and links the antibody unit to the Drug unit if the Stretcher unit and Spacer unit are absent.

-Ww- is a dipeptide, tripeptide, tetrapeptide, pentapeptide, hexapeptide, heptapeptide, octapeptide, nonapeptide, decapeptide, undecapeptide or dodecapeptide unit. The Amino Acid unit of the ADC of the invention can be enzymatically cleaved by one or more enzymes, including a tumor-associated protease, to liberate the Drug unit (-D). Illustrative Ww units are represented by formula;

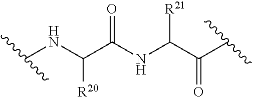

Wherein R²⁰ is isopropyl and R²¹ is (CH₂)₃NHCONH₂.

The Spacer unit: The Spacer unit (—Y—), when present, links an amino acid unit to the drug unit when an amino acid unit is present. Alternately, the Spacer unit links the Stretcher unit to the Drug unit when the amino acid unit is absent. The Spacer unit also links the Drug unit to the antibody unit when both the amino acid unit and stretcher unit are absent. Spacer units are of two general types: self-immolative and non-self-immolative. In one embodiment, the Spacer unit (—Y—) of the present invention can be self-immolative or non-self-immolative. A non-self-immolative Spacer unit is one in which part or all of the Spacer unit remains bound to the Drug unit after cleavage, particularly enzymatic, of an amino acid unit from the Drug-Linker-antibody conjugate. Examples of a non-self-immolative Spacer unit include, but are not limited to a (glycine-glycine) Spacer unit and a glycine Spacer unit. When a Compound of the invention containing a glycine-glycine Spacer unit or a glycine Spacer unit undergoes enzymatic cleavage via a tumor-cell associated-protease, a cancer-cell-associated protease or a lymphocyte-associated protease, a glycine-glycine-Drug moiety or a glycine-Drug moiety is cleaved from Ab-Aa-Ww-. In a one embodiment, —Y_{y}— is a p-aminobenzyl alcohol (PAB) unit. In one embodiment, the invention provides a Drug-Linker Compound or antibody-Linker drug conjugate in which the Spacer unit is absent (═O). In one embodiment, —Y— is a p-aminobenzyl alcohol (PAB) group that is linked to —W_{w}— via the amino nitrogen atom of the PAB group, and connected directly to -D via a carbonate, carbamate or ether group.

The drug unit: The present invention provides antibody drug conjugate wherein antibody is daratumumab and drug is auristatin analogues selected from MMAE and MMAF as defined by following formulas:

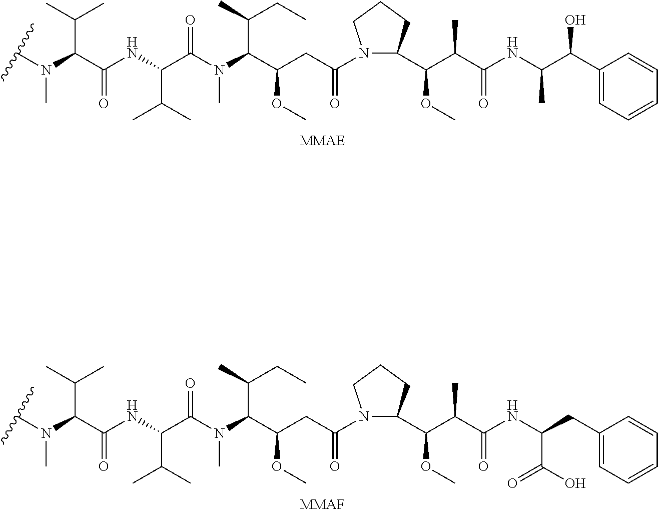

### Antibody Drug Conjugate of the Present Invention

The present invention provides antibody drug conjugate wherein the antibody is daratumumab or its variant that targets CD-38 antigen and drug or payload is an auristatin analogue preferably MMAE. The preferred antibody drug conjugate of the present invention is daratumumab-MMAE conjugate also named as daratumumab vedotin. The daratumumab-MMAE conjugate of the present invention comprises daratumumab or its variants as an antibody, mc-vc-PABC as a linker and MMAE as a toxin or drug or payload. The conjugate of the present invention also includes the pharmaceutical salt of the payload i.e. MMAE. The inventors of the present invention have found that the daratumumab-MMAE conjugates of the present invention produce significantly better biological activity in in-vitro as well as in-vivo studies in xenograft animal models as compared to an approved drug conjugate therapy (Darzalex®) in CD-38 expressing cancer. The novel drug conjugate of the present invention is better than the approved drug conjugate, Darzalex®.

It is expected that the biological activity of daratumumab-MMAE of the present invention will be extrapolated to clinical use in humans. The novel drug conjugate of the present invention is also better than the approved drug conjugate in that it is efficacious at significantly lower drug concentrations making the drug cheaper for the patients and possibly less toxic while being equally or better efficacious than the approved drug conjugate i.e. Darzalex®.

### Process of Preparing Antibody Drug Conjugate of the Present Invention:

Antibody of the present invention, preferably daratumumab was expressed in CHO (Chinese hamster ovary) cells using recombinant technology. Cells were cultured in cell culture media at suitable pH and temperature for specific period. After completion of cell incubation, cell cultures were harvested and antibody was isolated and purified by suitable chromatography technique. The antibody, preferably daratumumab, of the present invention was reduced with reducing agents such as Tris (2-carboxyethyl) phosphine (TCEP) in phosphate buffer at pH in the range of 5-8.5 at temperature about 37° C. for 120 minutes. Alternatively, daratumumab of the present invention can be reduced by dithiothreitol (DTT). The reduced daratumumab was reacted with about 5 to 10 molar excess of linker drug conjugate of the present invention. Linker drug conjugate of the present invention is vedotin, which is commercially available in the market. Vedotin of the present invention comprises mc-vc-PABC linker conjugated with monomethyl auristatin (MMAE) payload, also known as vc-MMAE or mc-vc-PABC-MMAE. The conjugate was purified by ultrafiltration-diafiltration. The drug antibody ratios (DAR) was determined by HPLC-HIC column method. The drug antibody ratios (DAR) obtained for daratumumab vedotin of the present invention, which were typically in the 1 to 8 range, preferably 3 to 5 more preferably 3.5 to 4.5. For long-term storage, the purified drug conjugate aliquots have been stored under frozen condition, at −25±5° C.

### Use of the Conjugate of the Present Invention:

In another aspect, the antibody drug conjugate of the present invention may be used in the treatment of a subject, comprising administering a therapeutically effective amount of a therapeutic conjugate, preferably daratumumab drug conjugate, as described herein to a subject. The effective dose of the conjugate may be in the range of 0.1 mg/kg to 10 mg/kg. Diseases that may be treated with the daratumumab drug conjugate include, but are not limited to, cancers, autoimmune diseases and/or infectious diseases, multiple myeloma, rheumatoid arthritis, polyarteritis nodosa, essential cryoglobulinaemic vasculitis, cutaneous leukocytoclastic angiitis, Kawasaki disease, Takayasu arteritis, giant cell arthritis, Henoch-Schonlein purpura, primary or isolated cerebral angiitis, erythema nodosum, thrombangiitis obliterans, thrombotic thrombocytopeniaurpura (including hemolytic uremic syndrome), and secondary vasculitides, including cutaneous leukocytoclastic vasculitis (e.g., secondary to hepatitis B, hepatitis C, Sjogren's syndrome, or systemic lupus erythematosus), erythema nodosum, allergic vasculitis, panniculitis, Weber-Christian disease, purpura hyperglobulinaemica, and Berger's disease, skin disorders, such as contact dermatitis, linear IgA dermatosis, vitiligo, pyoderma gangrenosum, epidermolysis bullosa acquisita, pemphigus vulgaris (including cicatricial pemphigoid and bullous pemphigoid), alopecia areata (including alopecia universalis and alopecia totalis), dermatitis herpetiformis, erythema multiforme, and chronic autoimmune urticaria (including angioneurotic edema and urticarial vasculitis), immune-mediated cytopenias, such as autoimmune neutropenia, and pure red cell aplasia; connective tissue disorders, such as CNS lupus, discoid lupus erythematosus, CREST syndrome, mixed connective tissue disease, polymyositis/dermatomyositis, inclusion body myositis, cryoglobulinemia type I and type II, fibromyalgia, phospholipid antibody syndrome, secondary hemophilia, relapsing polychondritis, sarcoidosis, stiff man syndrome, and rheumatic fever, eosinophil fasciitis, ankylosing spondylitis, juvenile chronic arthritis, adult Still's disease, and SAPHO syndrome, sacroileitis, reactive arthritis, Still's disease, gout; hematologic disorders, such as aplastic anemia, primary hemolytic anemia (including cold agglutinin syndrome), or systemic lupus erythematosus; POEMS syndrome, pernicious anemia, and Waldemstróm's purpura hyperglobulinaemica; further examples are agranulocytosis, autoimmune neutropenia, Franklin's disease, Seligmann's disease, gamma heavy chain disease, and factor VIII inhibitor formation; endocrinopathies, such as polyendocrinopathy, and Addison's disease; further examples are autoimmune hypoglycemia, autoimmune hypothyroidism, autoimmune insulin syndrome, de Quervain's thyroiditis, and insulin receptor antibody-mediated insulin resistance; hepato-gastrointestinal disorders, such as celiac disease, Whipple's disease, primary biliary cirrhosis, chronic active hepatitis, and primary sclerosing cholangiitis; a further example is autoimmune gastritis; nephropathies, such as rapid progressive glomerulonephritis, post-streptococcal nephritis, Goodpasture's syndrome, membranous glomerulonephritis, and cryoglobulinemic nephritis, minimal change disease; neurological disorders, such as autoimmune neuropathies, mononeuritis multiplex, Lambert-Eaton's myasthenic syndrome, Sydenham's chorea, tabes dorsalis, and Guillain-Barre's syndrome, myelopathy/tropical spastic paraparesis, myasthenia gravis, acute inflammatory demyelinating polyneuropathy, and chronic inflammatory demyelinating polyneuropathy; multiple sclerosis; cardiac and pulmonary disorders, such as COPD, fibrosing alveolitis, bronchiolitis obliterans, allergic aspergillosis, cystic fibrosis, Loffler's syndrome, myocarditis, and pericarditis; further examples are hypersensitivity pneumonitis, and allergic disorders, such as bronchial asthma and hyper-lgE syndrome, amaurosis fugax; ophthalmologic disorders, such as idiopathic chorioretinitis, infectious diseases, such as parvovirus B infection (including hands-and-socks syndrome); gynecological-obstretical disorders, such as recurrent abortion, recurrent fetal loss, and intrauterine growth retardation, paraneoplastic syndrome secondary to gynaecological neoplasms; male reproductive disorders, such as paraneoplastic syndrome secondary to testicular neoplasms.

Such therapeutics can be given once or repeatedly, depending on the disease state and tolerability of the conjugate, and can be used alone as a monotherapy or in combination with other therapeutic modalities, such as surgery, external radiation, radio immunotherapy, immunotherapy, chemotherapy, antisense therapy, interference RNA therapy, gene therapy, and the like. Each combination will be adapted to the tumor type, stage, patient condition and prior therapy, and other factors considered by the managing physician.

### Formulation and Administration

Suitable routes of administration of the daratumumab drug conjugates of the present invention include, but not limited to, oral, parenteral, rectal, transmucosal, intestinal administration, intramuscular, subcutaneous, intramedullary, intrathecal, direct intraventricular, intravenous, intravitreal, intraperitoneal, intranasal, or intraocular injections. The preferred routes of administration are parenteral. Alternatively, one may administer the compound in a local rather than systemic manner, for example, via injection of the compound directly into a solid tumor.

The antibody drug conjugate, preferably daratumumab drug conjugate of the present invention can be formulated according to known methods to prepare pharmaceutically useful compositions, whereby the antibody drug conjugate is combined in a mixture with a pharmaceutically suitable excipient. Sterile phosphate-buffered saline is one example of a pharmaceutically suitable excipient. Other suitable excipients are well known to those in the art.

In an embodiment, the present invention provides an antibody-linker-drug conjugates (ADC) of Formula (I):

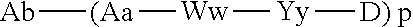

Wherein antibody (Ab) is a CD-38 targeting antibody;
-Aa-Ww-Yy- is an enzymatically cleavable linker unit that links the drug unit (D) and the antibody, wherein: -A- is a stretcher unit; a is 1; each —W— is independently an amino acid unit; —Y— is a spacer unit; w is an integer ranging from 2 to 12, y is 1 or 2; p ranges from 1 to about 20; and drug (D) is a suitable auristatin analogue selected from MMAE and MMAF as defined by following structures;

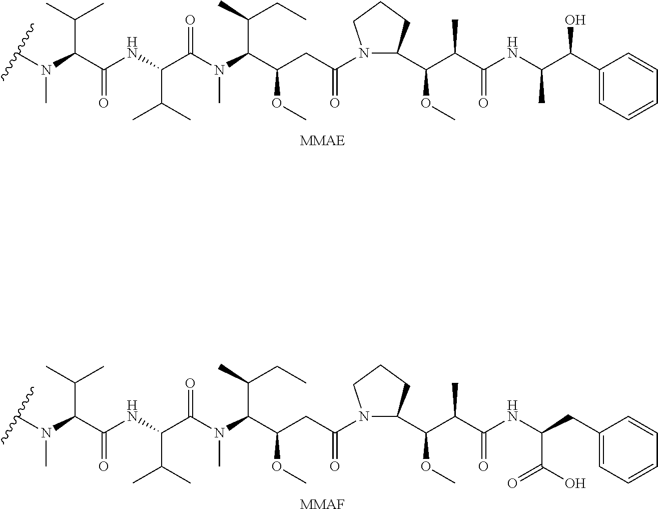

Wherein wavy line represent connection point to the linker.

In another embodiment, the present invention provides antibody-linker-drug conjugates wherein: -A- is a stretcher unit; —W— is independently an amino acid unit selected from -phenylalanine-lysine- or -valine-citrulline-; —Y— is a spacer unit selected from -glycine-glycine- or p-amino benzyl alcohol (PAB) or p-aminobenzyl carbamate (PABC).

In another embodiment, the present invention provides antibody-linker-drug conjugates wherein: -A- is a stretcher unit defined by following structure;

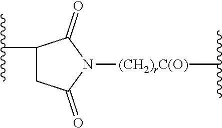

Wherein r is an integer ranging from 1 to 10; wherein carbonyl terminus of -A- forming bond with an amino acid unit (W) and the succinimido terminus of -A- forming bond with antibody (Ab).

In a yet another embodiment, the present invention provides antibody-linker-drug conjugates (ADC) wherein mAb is a monoclonal antibody comprising light chain variable region complementarity determining region (CDR) sequences CDR1 (RASQSVSSYLA, SEQ ID NO: 1), CDR2 (DASNRAT, SEQ ID NO: 2) and CDR3 (QQRSNWPPTF, SEQ ID NO: 3) and heavy chain variable region CDR sequences CDR1 (SFAMS, SEQ ID NO: 4), CDR2 (AISGSGGGTYYADSVKG, SEQ ID NO: 5) and CDR3 (DKILWFGEPVFDY, SEQ ID NO: 6); enzymatically cleavable linker unit is mc-vc-PABC and drug is drug (D) is a suitable auristatin analogue MMAE.

In a yet another embodiment, the present invention provides antibody-linker-drug conjugates (ADC) wherein the ADC structure is represented by;

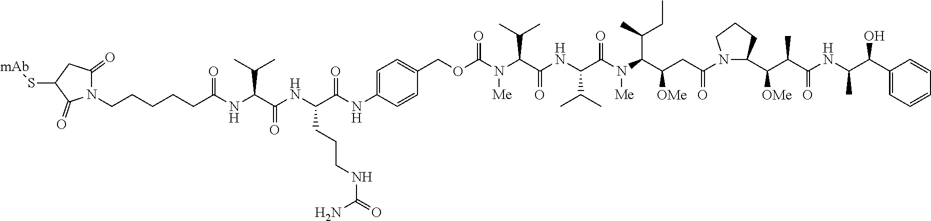

In another embodiment, the present invention provides mAb is a monoclonal antibody comprising light chain variable region complementarity determining region (CDR) sequences CDR1 (RASQSVSSYLA, SEQ ID NO: 1), CDR2 (DASNRAT, SEQ ID NO: 2) and CDR3 (QQRSNWPPTF, SEQ ID NO: 3) and heavy chain variable region CDR sequences CDR1 (SFAMS, SEQ ID NO: 4), CDR2 (AISGSGGGTYYADSVKG, SEQ ID NO: 5) and CDR3 (DKILWFGEPVFDY, SEQ ID NO: 6).

In an embodiment, the present invention provides antibody drug conjugate (ADC) as wherein mAb is a monoclonal antibody comprising constant domains and a hinge domain of a human IgG1 antibody.

In an embodiment, the present invention provides antibody drug conjugate (ADC) wherein mAb is a monoclonal antibody comprising constant domains and a hinge domain of a human IgG1 antibody and wherein one or more amino acids in the Fc portion are mutated.

In another embodiment, the present invention provides antibody drug conjugate (ADC) wherein mAb is the monoclonal antibody, Daratumumab or variants thereof.

In still another embodiment, the present invention provides antibody drug conjugate (ADC), wherein said mAb is attached to between 1 and 8 drug moieties, preferably 3 to 5, more preferably 3.5 to 4.5.

In an embodiment, the present invention provides a pharmaceutical composition comprising antibody drug conjugate (ADC) of the present invention and an acceptable carrier.

In an embodiment, the present invention provides a process for preparing the antibody drug conjugate of the present invention wherein process comprising a) purification of antibody, daratumumab b) partial reduction of daratumumab c) conjugation of reduced daratumumab with vc-MMAE drug linker.

In another embodiment, the present invention provides antibody drug conjugate (ADC), wherein said conjugate is in a form suitable for parenteral administration such as intravenous (i.v), intramuscular (i.m), subcutaneous (s.c.), intraperitoneal (i.p) or any other suitable route of administration.

In still another embodiment, the present invention provides antibody drug conjugate (ADC) wherein the ADC is used to treat cancer.

In yet another embodiment, the present invention provides antibody drug conjugate (ADC) wherein the ADC is used to treat an infection with a pathogenic organism.

In a further embodiment, the present invention provides antibody drug conjugate (ADC) wherein the ADC is used to treat an autoimmune disease.

In a still further embodiment, the present invention provides antibody drug conjugate (ADC) wherein the ADC is administered as monotherapy or in combination with one or more therapeutic agents selected from the group consisting of unconjugated antibodies, radiolabelled antibodies, drug-conjugated antibodies, toxin-conjugated antibodies, gene therapy, chemotherapy, therapeutic peptides, oligonucleotides, localized radiation therapy, surgery and interference RNA therapy.

In another embodiment, the present invention provides antibody drug conjugate (ADC) wherein the dose of the ADC is about 0.1 mg/kg to 10 mg/kg.

In an embodiment, the present invention provides that antibody drug conjugate (ADC) is daratumumab vedotin.

### Examples

### Example 1: Preparation of Daratumumab-Linker-Drug Conjugate

Daratumumab was expressed in CHO cells and purified by conventional column chromatography comprising r-Protein A affinity, strong cation exchange and mixed mode column chromatography. Purity of daratumumab was found to be 99.5%, as assessed by HP-SEC. Conjugation of drug to daratumumab (at >1 mg/mL) was performed on thiol (—SH) group under reducing conditions by molar excess (with respect to protein) of valine-citrulline coupled MMAE drug at temperature between 2° C. and 30° C. at pH between 5.0 and pH 8.5. The linker-drug complex was dissolved in 5-20% acetonitrile before mixing to the protein solution. The linker-drug complex can be also dissolved in other organic solvent like dimethyl acetate or dimethyl sulfoxide. The conjugation reaction was performed for about 60 minutes. Post-conjugation, the antibody drug conjugate (daratumumab-MMAE conjugate) was buffer exchanged, filtered through a 0.2μ filter and stored at or below −20° C. On HP-SEC analysis, the purity was observed as mentioned in table 1.

TABLE 1
Purity of daratumumab drug conjugate by HP-SEC analysis

| | HP-SEC analysis | | |
|---|---|---|---|
| Sample | HMW | Principal Peak | LMW |
| Daratumumab-MMAE conjugate | 3.89% | 95.00% | 1.11% |

The antibody drug conjugate was observed to show about 95% purity on HP-SEC analysis.

The Daratumumab-MMAE conjugate can also be prepared by method as described in Example 2.

### Example 2: Method of Preparing Daratumumab-MMAE Conjugate

Daratumumab monoclonal antibody was expressed in Chinese Hamster Ovary (CHO) cells. Purification was carried out using conventional column chromatography steps such as, r-Protein A affinity column chromatography, cation exchange chromatography and mixed-mode column chromatography. After purification, purity of the antibody was determined more than 99% using High-Performance Size Exclusion Chromatography (HP-SEC).

Conjugation of vcMMAE Drug to Daratumumab:
Purified Daratumumab monoclonal antibody was subjected to chemical conjugation with vc-MMAE. vc-MMAE matrix is also known as vedotin, which was purchased from market. The process steps are described as below;
   Thawing of the Critical Intermediate (CI, i.e. Daratumumab)

The CI was stored in frozen form in cryo-bags. Thawing was performed manually in the same cryo-bags. Upon thawing, the CI (Daratumumab) material was transferred to a suitable container for the next step of reaction.

### Partial Reduction of the CI by TCEP

Daratumumab was subjected to partial reduction (inter-chain S—S cross-links) in the presence of molar excess amount of tris (carboxyethyl) phosphine [TCEP] for about 300 min. Incubation temperature was maintained at or below 37° C., under stirring condition (200 rpm), for the reaction. At the end of reaction, the protein mixture was passed through a 0.2μ filter and collected in a clean de-pyrogenated glass bottle (2 L) to perform the next reaction step.

### Conjugation of the Drug-Linker to the CI

TCEP-treated Daratumumab was incubated with molar excess amount of the drug-linker, vc-MMAE (valine-citrulline monomethyl auristatin E). Incubation was performed under cold condition for NMT 120 min, with gentle stirring (200 rpm). Conjugation takes place with the formation of covalent thioether bond between the free-SH groups (inter-chains) of partially reduced Daratumumab protein and maleimide group of the drug linker, vcMMAE. At the end of conjugation reaction, the reaction mixture was passed through a 0.2 μm filter and subjected to UF/DF for the removal of unreacted drug-linker molecules.

### Removal of Excess Drug-Linker and Buffer Exchange by UF/DF

Post-filtration, the crude reaction mixture containing Daratumumab vedotin was subjected to UF/DF for the removal of unreacted vcMMAE drug-linker, followed by final buffer exchange. For UF/DF, 30 kDa MWCO membrane filter were used. UF/DF was carried out at a maximum transmembrane pressure (TMP) of 0.50 bar. Constant volume diafiltration was carried out with 20 mM Na-citrate buffer of pH 6.6±0.1; conductivity 4.5±0.5 mS/cm, for maximum 30 dia-volumes. Diafiltration was monitored and controlled for pH and conductivity of the retentate to achieve the target values. After achieving the target pH and conductivity, Daratumumab vedotin was recovered at about 7 mg/mL, and passed through a 0.2μ filter.

### Preparation of Formulated Bulk

The concentrated protein solution was collected in a clean depyrogenated glass bottle. To it, trehalose was added from freshly prepared 30% stock solution (0.2μ filtered) to achieve a final concentration of 7% (w/v). Subsequently, polysorbate 80 was added to a final concentration of 0.02% and the final volume of bulk was adjusted with addition of the drug substance buffer to achieve a protein concentration of 5 mg/mL for Daratumumab vedotin. The formulated bulk of Daratumumab vedotin was passed through a 0.2 μm filter, under bio-safety cabinet, and collected in single-use, sterile, non-pyrogenic polyethylene terephthalate glycol (PETG) bottle with white high-density polyethylene (HDPE) screw closure. The filtered formulated bulk was labeled as the Daratumumab vedotin drug substance.

### Example 3: Drug Antibody Ratio (DAR) of Daratumumab-MMAE Conjugate

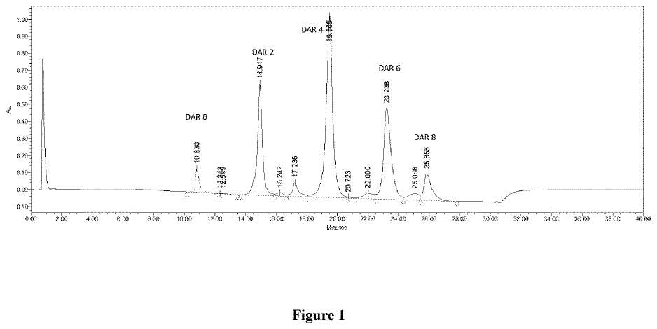

Daratumumab vedotin drug substance samples of example 3 were analyzed by analytical Hydrophobic Interaction Chromatography (HIC)-HPLC to assess the distribution of drug-linker (Dl) on Daratumumab. The peaks are separated by HIC on the basis of relative hydrophobicity of the drug-conjugated-antibody (ADC) carrying 0 to 8 payloads (drug toxin). This technique also allows the determination of average drug-to-antibody ratio (DAR) in Daratumumab vedotin. Experiments were performed by using a butyl (4.6×100 mm; 5 μm) column coupled with HPLC system. Samples containing Daratumumab vedotin drug substance were injected onto the HIC column that was saturated with mobile phase A (50 mM phosphate pH 7.0 and 1.2 M ammonium sulfate buffer containing 5% iso-propyl alcohol), at 25° C. Daratumumab vedotin carrying varying number of payloads were eluted out of the column with mobile phase B (50 mM phosphate of pH 7.0 containing 20% iso-propyl alcohol), in decreasing salt concentration mode, at 1.0 mL/min, and elution was recorded with UV detection, at 214 nm. FIG. 1 illustrates the appearance of drug-linked antibody variants obtained with three independent batches of Daratumumab vedotin.

All three Daratumumab vedotin drug substance samples were found to show five prominent peaks, which correspond to DAR0, DAR2, DAR4, DAR6 and DAR8, as shown in FIG. 1. Retention time (RT) values of the individual peaks obtained with the batch samples were observed to be nearly identical to each other. Daratumumab vedotin obtained from all the batches derived were showing an average DAR 4±1, preferably DAR about 1 to 8 more preferably about 3.5 to 4.5 on HP—HIC analysis as provided in below table 2 and FIG. 1.

TABLE 2
% Drug antibody ratio (DAR) of Daratumumab-MMAE conjugate

| | % DAR | % DAR | % DAR | % DAR | % DAR | Average |
|---|---|---|---|---|---|---|
| Samples | 0 | 2 | 4 | 6 | 8 | DAR |
| Daratumumab- | 3.36 | 20.64 | 43.18 | 25.16 | 7.66 | 4.26 |
| MMAE | | | | | | |
| conjugate | | | | | | |

### Example 4: Polypeptide Profile of Daratumumab-Linker-Drug Conjugate Using SDS PAGE Electrophoresis.

Preparation of daratumumab-linker-drug conjugate: Daratumumab-linker-drug conjugate was prepared according to process as described in Example 1.

### SDS PAGE Electrophoresis:

Instrument: BIO-RAD electrophoresis System.

Electrophoresis Buffer composition: 0.0247 M Tris, 0.192 M glycine, 0.1% SDS, pH 8.3±0.1.

Sample Preparation: Each sample along with Internal Reference standard was diluted with Milli-Q water and 5× sample buffer in such a way that, while loading sample each gel well will be loaded with 10 μg protein.

### Polypeptide Profile of Daratumumab-Linker-Drug Conjugate (Daratumumab ADC):

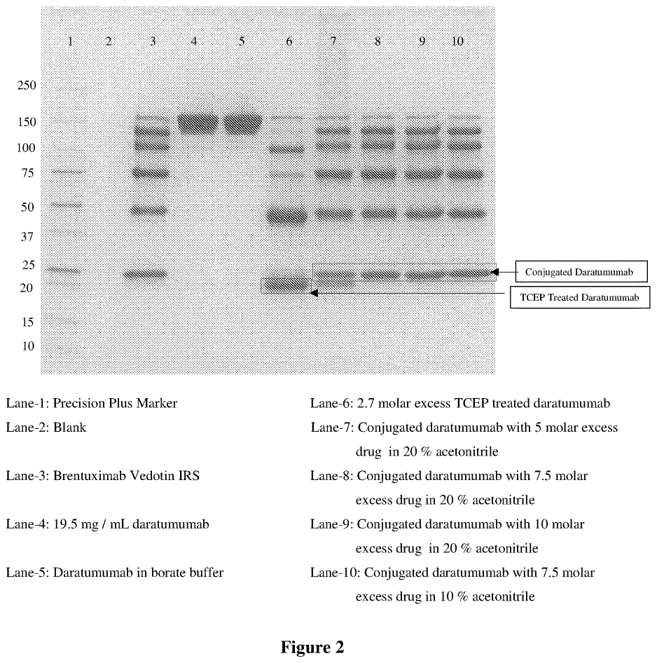

Polypeptide profile of daratumumab ADC has been demonstrate in FIG. 2. It describes the results of SDS-PAGE analyses of various samples obtained during conjugation reaction of daratumumab ADC, including the purified daratumumab in lane no 4 and 5. SDS-PAGE analyses of the samples were performed under non-reducing conditions. Internal reference standard i.e. brentuximab vedotin (ADC) was also analysed under the same conditions, as positive control. Post SDS-PAGE analyses, it has been observed that the daratumumab is intact in borate buffer after buffer exchange as single band is observed in lane no 4 and 5. Lane no 6 of non-reducing SDS-PAGE shows multiple bands of heterogenous species of mAb post partial reduction with 2.7 Molar TCEP. The conjugated sample in lane no 8, 9 and 10 signifies the successful conjugation as each band with payload shifts towards upper side i.e. higher molecular weight compared to TCEP treated sample in lane no 6.

### Example 5: In-Vitro Cytotoxicity Study of Daratumumab-Linker-Drug Conjugate

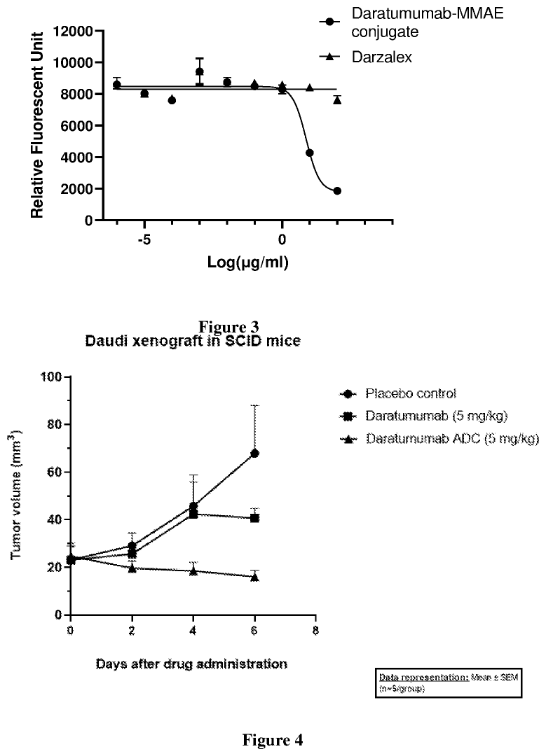

Daudi (ATCC CCL-213, B Lymphoblast) cells were seeded on a 96 well black clear bottom plate (Costar 3603). The cell line was chosen as it is known to express the CD38 receptor. The cells were treated with different concentrations of daratumumab-MMAE conjugation and Darzalex, incubated in CO₂ incubator for 2 hours. After incubation the excess or unbound Daratumumab-MMAE was removed, fresh media was added and incubated for 4 days in 5% CO₂ at 37° C. At the end of incubation, alamar blue dye (invitrogen, DAL-1100) was added to the cells and incubated for 5 hours in CO₂ incubator. After incubation viable cells were quantified by measuring the fluorescence signals at an excitation wavelength of 530 nm and an emission wavelength of 590 nm using a SpectraMax M2E fluorescent plate reader FIG. 3 shows that the relative in vitro activity of the tested daratumumab-MMAE conjugate is better than Darzalex. The Daratumumab antibody alone does not show activity without effector functions. However, the daratumumab-MMAE conjugate shows good in vitro activity without the requirement of effector cell mediated killing. Accordingly, data shows that the daratumumab-MMAE conjugate of the present invention is active and non-inferior to Darzalex.

### Example 6: Efficacy of Daratumumab-ADC in Burkitt Lymphoma Xenograft Disease Model in SCID Mice

In present study, the therapeutic potential of novel Daratumumab-ADC has been evaluated in comparison to Daratumumab antibody only. Specifically to induce Burkitt lymphoma xenograft, Immunodeficient SCID mice were injected with Daudi cells (1 million cells/animal/200 μL volume, In 1:1 ratio of cell suspension & matrigel) in right flank region of animals. Animals were observed for tumor development & drug administration was done when mean tumor volume of animals were reached to approximately 20-25 mm3. Here, in this study, Daratumumab-ADC & Daratumumab were compared head to head at a dose level of 5 mg/kg (I.V. route, Single administration).

FIG. 4 shown below demonstrate efficacy of Daratumumab-ADC in comparison to Daratumumab in Daudi Burkitt lymphoma cell line. Here, Daratumumab at a dose of 5 mg/kg were able to delay the tumor progression in comparison to placebo control group but not able to regress the tumor burden. At the same time, Daratumumab-ADC at a similar dose were able to regress the tumor burden, which indicates that Daratumumab-ADC has good potential to regress burkitt lymphoma tumor carrying CD38 over-expression and has demonstrated clear superiority over available Anti-CD38 therapy Daratumumab monoclonal antibody.

### REFERENCES INCORPORATED IN CURRENT PATENT APPLICATION

1. Diamantis N, Banerji U. Antibody—drug conjugate an emerging class of cancer treatment. Br J Cancer 2015; 114: 362-367.
2. Bouchard H, Viskov C, Garcia-Echeverria C. Antibody-drug conjugates—a new wave of cancer drugs. Bioorganic Med. Chem Lett 2014; 24: 5357-5363.
3. Tolcher A W, Ann Oncol. 2016; 27: 2168-2172. 10.1093/annonc/mdw424. [PubMed: 27733376]
4. Lokhorst H M, Plesner T, Laubach J P, Nahi H, Gimsing P, Hansson M, Minnema M C, Lassen U, Krejcik J, Palumbo A, van de Donk N W C J, Ahmadi T, Khan I, Uhlar C M, Wang J, Sasser A K, Losic N, Lisby S, Basse L, Brun N and Richardson P G, N Engl J Med. 2015; 373: 1207-1219. 10.1056/NEJMoa1506348. [PubMed: 26308596]
5. de Weers M, Tai Y-T, van der Veer M S, Bakker J M, Vink T, Jacobs D C H, Oomen L A, Peipp M, Valerius T, Slootstra J W, Mutis T, Bleeker W K, Anderson K C, Lokhorst H M, van de Winkel J G J and Parren P W H I, J Immunol. 2011; 186: 1840-1848. 10.4049/jimmunol. 1003032. [PubMed: 21187443]
6. Malavasi, F., A. Funaro, S. Roggero, A. Horenstein, L. Calosso, and K. Mehta. 1994. Human CD38: a glycoprotein in search of a function. Immunol. Today 15: 95-97.
7. Mehta, K., and F. Malavasi, eds. 2000. Human CD38 and Related Molecules. Karger, Basel, Switzerland.
8. WHO Drug Information, Vol. 24, No. 1, 2010 Recommended INN: List 63 41 International Non-proprietary Names for Pharmaceutical Substances (INN).

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

| | | |
|---|---|---|
| 1 | Sequence Listing Information | |
| 1-1 | File Name | ZLL-PCT0890.xml |
| 1-2 | DTD Version | V1_3 |
| 1-3 | Software Name | WIPO Sequence |
| 1-4 | Software Version | 2.3.0 |
| 1-5 | Production Date | 2024-03-15 |
| 1-6 | Original free text language code | |
| 1-7 | Non English free text language | |
| | code | |
| 2 | General Information | |
| 2-1 | Current application: IP Office | IN |
| 2-2 | Current application: Application | 202321018199 |
| | number | |
| 2-3 | Current application: Filing date | 2023-03-17 |
| 2-4 | Current application: Applicant | ZLL-PCT0890 |
| | file reference | |
| 2-5 | Earliest priority application: IP | IN |
| | Office | |
| 2-6 | Earliest priority application: | 202321018199 |
| | Application number | |
| 2-7 | Earliest priority application: | 2023-03-17 |
| | Filing date | |
| 2-8en | Applicant name | ZYDUS LIFESCIENCES LIMITED |
| 2-8 | Applicant name: Name Latin | |
| 2-9 | Inventor name | |
| 2-9 | Inventor name: Name Latin | |
| 2-10en | Invention title | Daratumumab drug conjugates and preparation |
| | | thereof |

| | | |
|---|---|---|
| 2-11 | Sequence Total Quantity | 8 |

| | | |
|---|---|---|
| 3-1 | Sequences | |
| 3-1-1 | Sequence Number [ID] | 1 |
| 3-1-2 | Molecule Type | AA |
| 3-1-3 | Length | 11 |
| | | REGION 1..11 |
| 3-1-4-1 | Features Location/Qualifiers | note = Light chain variable region comp- |
| | | lementarity determining region |
| | | (CDR) sequence CDR1 |
| | NonEnglishQualifier Value | source 1..11 |
| 3-1-4-2 | Features Location/Qualifiers | mol_type = protein |
| | | organism = synthetic construct |
| | NonEnglishQualifier Value | |
| 3-1-5 | Residues | |

| | |
|---|---|
| RASQSVSSYL A | 11 |

| | | |
|---|---|---|
| 3-2 | Sequences | |
| 3-2-1 | Sequence Number [ID] | 2 |
| 3-2-2 | Molecule Type | AA |
| 3-2-3 | Length | 7 |
| | | REGION 1..7 |
| 3-2-4-1 | Features Location/Qualifiers | note = Light chain variable region comp- |
| | | lementarity determining region |
| | | (CDR) sequence CDR2 |
| | NonEnglishQualifier Value | source 1..7 |
| | | mol_type = protein |
| 3-2-4-2 | Features Location/Qualifiers | organism = synthetic construct |
| | NonEnglishQualifier Value | |
| 3-2-5 | Residues | |

| | |
|---|---|
| DASNRAT | 7 |

| | | |
|---|---|---|
| 3-3 | Sequences | |
| 3-3-1 | Sequence Number [ID] | 3 |
| 3-3-2 | Molecule Type | AA |
| 3-3-3 | Length | 10 |
| | | REGION 1..10 |
| 3-3-4-1 | Features Location/Qualifiers | note = Light chain variable region comp- |
| | | lementarity determining region |
| | | (CDR) sequence CDR3 |
| | NonEnglishQualifier Value | source 1..10 |
| 3-3-4-2 | Features Location/Qualifiers | mol_type = protein |
| | | organism = synthetic construct |
| | NonEnglishQualifier Value | |
| 3-3-5 | Residues | |

| | |
|---|---|
| QQRSNWPPTF | 10 |

| | | |
|---|---|---|
| 3-4 | Sequences | |
| 3-4-1 | Sequence Number [ID] | 4 |
| 3-4-2 | Molecule Type | AA |
| 3-4-3 | Length | 5 |
| | | REGION 1..5 |
| 3-4-4-1 | Features Location/Qualifiers | note = Heavy chain variable region comp- |
| | | lementarity determining region |
| | | (CDR) sequence CDR1 |
| | NonEnglishQualifier Value | source 1..5 |
| | | mol_type = protein |
| 3-4-4-2 | Features Location/Qualifiers | organism = synthetic construct |
| | NonEnglishQualifier Value | |
| 3-4-5 | Residues | |

| | |
|---|---|
| SFAMS | 5 |

| | | |
|---|---|---|
| 3-5 | Sequences | |
| 3-5-1 | Sequence Number [ID] | 5 |
| 3-5-2 | Molecule Type | AA |
| 3-5-3 | Length | 17 |
| | | REGION 1..17 |
| 3-5-4-1 | Features Location/Qualifiers | note = Heavy chain variable region comp- |
| | | lementarity determining region |
| | | (CDR) sequence CDR2 |
| | NonEnglishQualifier Value | |
| 3-5-4-2 | Features Location/Qualifiers | source 1..17 |
| | | mol_type = protein |
| | | organism = synthetic construct |
| | NonEnglishQualifier Value | |
| 3-5-5 | Residues | |

| | |
|---|---|
| AISGSGGGTY YADSVKG | 17 |

| | | |
|---|---|---|
| 3-6 | Sequences | |
| 3-6-1 | Sequence Number [ID] | 6 |
| 3-6-2 | Molecule Type | AA |
| 3-6-3 | Length | 13 |
| | | REGION 1..13 |
| 3-6-4-1 | Features Location/Qualifiers | note = Heavy chain variable region comp- |
| | | lementarity determining region |
| | | (CDR) sequence CDR3 |
| | NonEnglishQualifier Value | source 1..13 |
| 3-6-4-2 | Features Location/Qualifiers | mol_type= protein |
| | | organism= synthetic construct |
| | NonEnglishQualifier Value | |
| 3-6-5 | Residues | |

| | |
|---|---|
| DKILWFGEPV FDY | 13 |

| | | |
|---|---|---|
| 3-7 | Sequences | |
| 3-7-1 | Sequence Number [ID] | 7 |
| 3-7-2 | Molecule Type | AA |
| 3-7-3 | Length | 214 |
| 3-7-4-1 | Features Location/Qualifiers | REGION 1..214 |
| | | note = Light chain sequence of CD-38 |
| | | targeting antibody |
| | NonEnglishQualifier Value | source 1..214 |
| 3-7-4-2 | Features Location/Qualifiers | mol_type = protein |
| | | organism = synthetic construct |
| | NonEnglishQualifier Value | |
| 3-7-5 | Residues | |

| | |
|---|---|
| EIVLTQSPAT LSLSPGERAT LSCRASQSVS SYLAWYQQKP GQAPRLLIYD ASNRATGIPA | 60 |
| RFSGSGSGTD FTLTISSLEP EDFAVYYCQQ RSNWPPTFGQ GTKVEIKRTV AAPSVFIFPP | 120 |
| SDEQLKSGTA SVVCLLNNFY PREAKVQWKV DNALQSGNSQ ESVTEQDSKD STYSLSSTLT | 180 |
| LSKADYEKHK VYACEVTHQG LSSPVTKSFN RGEC | 214 |

| | | |
|---|---|---|
| 3-8 | Sequences | |
| 3-8-1 | Sequence Number [ID] | 8 |
| 3-8-2 | Molecule Type | AA |
| 3-8-3 | Length | 452 |
| 3-8-4-1 | Features Location/Qualifiers | REGION 1..452 |
| | | note = Heavy chain sequence of CD-38 |
| | | targeting antibody |
| | NonEnglishQualifier Value | |
| | | source 1..452 |
| 3-8-4-2 | Features Location/Qualifiers | mol_type = protein |
| | | organism = synthetic construct |
| | NonEnglishQualifier Value | |
| 3-8-5 | Residues | |

| | |
|---|---|
| EVQLLESGGG LVQPGGSLRL SCAVSGFTFN SFAMSWVRQA PGKGLEWVSA ISGSGGGTYY | 60 |
| ADSVKGRFTI SRDNSKNTLY LQMNSLRAED TAVYFCAKDK ILWFGEPVED YWGQGTLVTV | 120 |
| SSASTKGPSV FPLAPSSKST SGGTAALGCL VKDYFPEPVT VSWNSGALTS GVHTFPAVLQ | 180 |
| SSGLYSLSSV VTVPSSSLGT QTYICNVNHK PSNTKVDKRV EPKSCDKTHT CPPCPAPELL | 240 |
| GGPSVFLFPP KPKDTLMISR TPEVTCVVVD VSHEDPEVKF NWYVDGVEVH NAKTKPREEQ | 300 |
| YNSTYRVVSV LTVLHQDWLN GKEYKCKVSN KALPAPIEKT ISKAKGQPRE PQVYTLPPSR | 360 |
| EEMTKNQVSL TCLVKGFYPS DIAVEWESNG QPENNYKTTP PVLDSDGSFF LYSKLTVDKS | 420 |
| RWQQGNVFSC SVMHEALHNH YTQKSLSLSP GK | 452 |

1. An antibody-linker-drug conjugate (ADC) comprising a monoclonal antibody (mAb) comprising light chain variable region complementarity determining region (CDR) sequences CDR1 (RASQSVSSYLA, SEQ ID NO: 1), CDR2 (DASNRAT, SEQ ID NO: 2), and CDR3 (QQRSNWPPTF, SEQ ID NO: 3) and heavy chain variable region CDR sequences CDR1 (SFAMS, SEQ ID NO: 4), CDR2 (AISGSGGGTYYADSVKG, SEQ ID NO: 5), and CDR3 (DKILWFGEPVFDY, SEQ ID NO: 6); a linker comprising an enzymatically cleavable linker unit; and a drug, wherein the enzymatically cleavable linker unit comprises mc-vc-PABC and wherein the drug is an auristatin analogue MMAE.

2. The ADC of claim 1, wherein the ADC structure is:

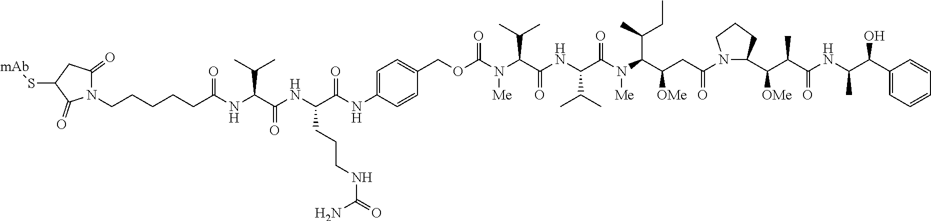

3. The ADC of claim 1, wherein the mAb comprises constant domains and a hinge domain of a human IgG1 antibody.

4. The ADC of claim 3, wherein one or more amino acids in the Fc portion are mutated.

5. The ADC of claim 3, wherein the mAb is daratumumab, or a variant thereof.

6. The ADC of claim 1, wherein the mAb is attached to between 1 and 8 drug moieties.

7. The ADC of claim 1, wherein the mAb is attached to 3.50, 3.51, 3.52, 3.53, 3.54, 3.55, 3.56, 3.57, 3.58, 3.59, 3.60, 3.61, 3.62, 3.63, 3.64, 3.65, 3.66, 3.67, 3.68, 3.69, 3.70, 3.71, 3.72, 3.73, 3.74, 3.75, 3.76, 3.77, 3.78, 3.79, 3.80, 3.81, 3.82, 3.83, 3.84, 3.85, 3.86, 3.87, 3.88, 3.89, 3.90, 3.91, 3.92, 3.93, 3.94, 3.95, 3.96, 3.97, 3.98, 3.99, 4.00, 4.01, 4.02, 4.03, 4.04, 4.05, 4.06, 4.07, 4.08, 4.09, 4.10, 4.11, 4.12, 4.13, 4.14, 4.15, 4.16, 4.17, 4.18, 4.19, 4.20, 4.21, 4.22, 4.23, 4.24, 4.25, 4.26, 4.27, 4.28, 4.29, 4.30, 4.31, 4.32, 4.33, 4.34, 4.35, 4.36, 4.37, 4.38, 4.39, 4.40, 4.41, 4.42, 4.43, 4.44, 4.45, 4.46, 4.47, 4.48, 4.49, or 4.50 drug moieties.

8. A pharmaceutical composition comprising the ADC of claim 1 and a pharmaceutically acceptable carrier.

9. A process for preparing the ADC of claim 5, wherein the process comprises a) purifying daratumumab, b) partially reducing daratumumab, and c) conjugating the partially reduced daratumumab with a vc-MMAE drug linker.

10. The method of claim 14, wherein the ADC is administered parenterally.

11. A method for treating a cancer in a subject in need thereof, comprising administering to the subject a therapeutically effective amount of the ADC of claim 1.

12. A method for treating an infection caused by a pathogenic organism in a subject in need thereof, comprising administering to the subject a therapeutically effective amount of the ADC of claim 1.

13. A method for treating an autoimmune disease in a subject in need thereof, comprising administering to the subject a therapeutically effective amount of the ADC of claim 1.

14. A method for treating a disease in a subject in need thereof, comprising administering to the subject a therapeutically effective amount of the ADC of claim 1, wherein the ADC is administered as a monotherapy or in combination with one or more therapeutic agents selected from the group consisting of unconjugated antibodies, radiolabelled antibodies, drug-conjugated antibodies, toxin-conjugated antibodies, gene therapy, chemotherapy, therapeutic peptides, oligonucleotides, localized radiation therapy, surgery, and interference RNA therapy.

15. The method of claim 14, wherein the ADC is administered in an amount from about 0.1 mg/kg to about 10 mg/kg.

16. The ADC of claim 1, wherein the mAb is daratumumab and the ADC structure is:

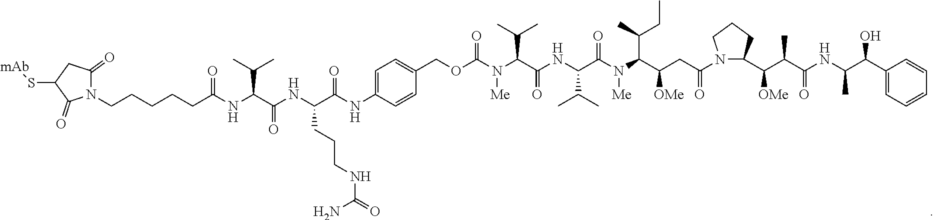

17. The ADC of claim 6, wherein the mAb is attached to between 3 and 5 drug moieties.

18. The ADC of claim 6, wherein the mAb is attached to between 3.5 and 4.5 drug moieties.

19. The method of claim 10, wherein the ADC is administered intravenously, intramuscularly, subcutaneously, or intraperitoneally.

20. A pharmaceutical dosage form comprising the ADC of claim 1, wherein the dosage form is suitable for parenteral administration.


SEQUENCE LISTING (8 sequences)

SEQ ID NO: 1 - protein, 11 aa, synthetic construct
  REGION 1..11: note = Light chain variable region complementarity determining region (CDR) sequence CDR1
RASQSVSSYLA

SEQ ID NO: 2 - protein, 7 aa, synthetic construct
  REGION 1..7: note = Light chain variable region complementarity determining region (CDR) sequence CDR2
DASNRAT

SEQ ID NO: 3 - protein, 10 aa, synthetic construct
  REGION 1..10: note = Light chain variable region complementarity determining region (CDR) sequence CDR3
QQRSNWPPTF

SEQ ID NO: 4 - protein, 5 aa, synthetic construct
  REGION 1..5: note = Heavy chain variable region complementarity determining region (CDR) sequence CDR1
SFAMS

SEQ ID NO: 5 - protein, 17 aa, synthetic construct
  REGION 1..17: note = Heavy chain variable region complementarity determining region (CDR) sequence CDR2
AISGSGGGTYYADSVKG

SEQ ID NO: 6 - protein, 13 aa, synthetic construct
  REGION 1..13: note = Heavy chain variable region complementarity determining region (CDR) sequence CDR3
DKILWFGEPVFDY

SEQ ID NO: 7 - protein, 214 aa, synthetic construct
  REGION 1..214: note = Light chain sequence of CD-38 targeting antibody
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 452 aa, synthetic construct
  REGION 1..452: note = Heavy chain sequence of CD-38 targeting antibody
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK